(19) United States
(12) Patent Application Publication
(10) Pub. No.: US 2018/0214130 A1
(43) Pub. Date: Aug. 2, 2018

(54) COMPACT FINGERTIP-MANIPULATED ULTRASOUND IMAGING

(71) Applicant: University of Virginia Patent Foundation, Charlottesville, VA (US)

(72) Inventors: John A. Hossack, Charlottesville, VA (US); Gorav Ailawadi, Charlottesville, VA (US)

(21) Appl. No.: 15/748,555

(22) PCT Filed: Jul. 28, 2016
(86) PCT No.: PCT/US16/44498

§ 371 Date: Jan. 29, 2018

Related U.S. Application Data
(60) Provisional application No. 62/199,103, filed on Jul. 30, 2015.

(51) Int. Cl. A61B 8/00; A61B 8/14; A61B 8/08

(52) U.S. Cl. CPC A61B 8/467; A61B 8/4209; A61B 8/462; A61B 8/4427; A61B 8/4472; A61B 2090/502; A61B 8/145; A61B 8/54; A61B 8/4488; A61B 8/56; A61B 8/0891; A61B 8/4455

(57) ABSTRACT

Aspects of various examples described herein can include using a wearable ultrasound device (e.g., a device wearable or manipulated using a fingertip). The wearable ultrasound device can include an ultrasound transducer element array, and can have a housing providing circuitry to process signals to or from the array, such as digitizing such signals. For example, a channel count of the analog signal channels corresponding to individual sensors in the array can be converted to a single digital channel (e.g., a serial channel), or at least a reduced channel count as compared to a count of transducer elements. In this manner, a wired or wireless coupling between the ultrasound “front end” circuitry in the wearable device and processing or display hardware elsewhere need not accommodate a large number of analog channels, and can instead, for example, have a digital communication interface.

### CLAIM OF PRIORITY

This patent application claims the benefit of priority to Hossack et al., U.S. Provisional Patent Application No. 62,199/103, titled “Compact Finger Tip Ultrasound System and Related Methods,” filed on Jul. 30, 2015, the entirety of which is hereby incorporated herein by reference.

### BACKGROUND

Coronary Artery Bypass Graft (CABG) may be regarded as the most commonly encountered open heart cardiothoracic surgical operation in the United States of America. Approximately 395,000 CABG procedures are performed each year in the United States. Although the rate of CABG procedures has been in slow decline with the advent of Percutaneous Coronary Intervention (PCI), the decline may be regarded as reaching stabilization. For example, a significant proportion of patients who possess Coronary Artery Disease (CAD) are unsuited to PCI. Specifically, cases involving multi-vessel disease and left main coronary artery disease are more appropriately treated using a CABG as opposed to PCI. Rates for reocclusion using CABG are lower in comparison to re-occlusion rates involving PCI. Over time, as the patient population ages, there will be growing numbers of more complex coronary cases (e.g. increasing incidences of re-occlusion of previous PCI or CABG, multi-vessel disease, diabetic patients with propensity to more aggressive CAD, or other complex cases) for which open chest surgery, or minimally invasive cardiothoracic surgery, is the best, or even only, option.

CABG procedures are very time and surgical skill intensive. Total OR duration may be of the order of four hours—depending on complexity of the case. This results in very high patient and societal costs. Additionally, procedure duration (especially that portion of the procedure conducted while the patient is on cardiopulmonary bypass) have implications for patient mortality and morbidity (i.e. cardio-pulmonary bypass is associated with memory loss, risk of stroke, etc.)

### SUMMARY

Aspects of various examples described herein can include using a wearable ultrasound device (e.g., a device wearable or manipulated using a fingertip). The wearable ultrasound device can include an ultrasound transducer element array, and can have a housing providing circuitry to process signals to or from the array, such as digitizing such signals. For example, a channel count of the analog signal channels corresponding to individual sensors in the array can be converted to a single digital channel (e.g., a serial channel), or at least a reduced channel count as compared to a count of transducer elements. In this manner, a wired or wireless coupling between the ultrasound “front end” circuitry in the wearable device and processing or display hardware elsewhere need not accommodate a large number of analog channels, and can instead, for example, have a digital communication interface. The wearable ultrasound device can include a disposable housing, or can be disposable in its entirety, such as to eliminate costly sterilization or handling steps. Generally, cleaning (e.g., removal of any form of liquid or solid contamination) and sterilization (e.g., killing any microbial residue) can be problematic and expensive. For example, cleaning and sterilization of tight internal corners is occasionally incomplete. The most effective cleaning and sterilization techniques are also generally aggressive and harmful to the materials of the component being cleaned or sterilized. Thus, the costs and number of viable clean / sterilize cycles frequently make re-use of components impractical and cost-prohibitive. In the case of a “single use” wearable ultrasound device, the device can be used in a single instance for a surgical or diagnostic procedure, and can then be discarded.

In an illustrative example, a single-use finger-tip mounted ultrasound array can include “front end” processing in the tip and low-cost, low-channel-count, wire interface to an imaging base station. A primary clinical application can include cardiothoracic surgery and, in particular, procedures involving Internal Thoracic Artery (ITA) harvest and bypass grafting. There are 395,000 Coronary Artery Bypass Graft (CABG) procedures performed each year in the US. The present inventors forecast, that due to obscuring tissue, 20% of these cases can benefit from cross-sectional imaging guidance. Additional surgical applications of the device may also be applicable.

Generally, a wearable ultrasound device can facilitate one or more of the following operations:

1. Finding and guiding resection (e.g., surgical “cut down” /isolation) of the Internal Thoracic Artery (ITA)

2. Finding ITA branches and estimating their size. As an illustrative example, such as during ITA harvest, it is helpful to know of the location and size of branches. Larger branches generally require surgical tie-off and smaller branches can be cauterized to seal them off. Use of a fingertip mounted or fingertip manipulated ultrasound device can facilitate such location and estimation involving ITA branches.

3. Verifying the quality of blood flow in coronaries post procedure, such as using Doppler imaging techniques or echogenic contrast agents.

4. Finding the ITA, or any other vessel, in the case of a surgical “re-do.” In a “re-do,” the anatomy may be unknown due to the relocation of vessels from their original location. Vessels of interest may be severely obscured by layers of scar or fascia tissue formed subsequent to previous surgery. In the context of an aging population and higher expectations for surgical solutions to previously addressed problems, the number of “re-do” operations is projected to increase as a proportion of the total number of CABG related operations.

5. Generic vessel finding. In the context of many procedures (e.g. minimally invasive procedures—e.g. PCI) blood vessel location is used to provide for vascular access (needle or catheter). For example, PCI is migrating to more widespread use of radial arteries and these can be more difficult to find than the femoral artery in the crotch region.

6. Other surgical procedures not specifically related to CABG or PCI, such as neck, vascular, organ, cancer-related or emergency procedures.

In an example, an ultrasound imaging system can include a wearable ultrasound device. The wearable ultrasound device can include an ultrasound transducer array, a wearable clip mechanically coupled to the transducer array, a housing including an ultrasound transmit-and-receive circuit electrically coupled to the transducer array, and a digital communication circuit coupling the ultrasound transmit-and-receive circuit. The digital communication circuit can be configured to provide a digital representation indicative of receive ultrasound echo information. The digital coupling can be electrical or optical-based and may be serial, parallel or a combination of the two. The wearable clip can be configured to securely anchor the wearable ultrasound device assembly to a finger of a user, and the ultrasound transducer array, wearable clip, and housing are generally sized and shaped to be lifted and manipulated by as few as a single finger of a user.

In an example, the ultrasound imaging system can include a display coupled to a control circuit to receive the digital representation indicative of received ultrasound echo information. The display can be integrated with the housing or separate from the housing of the wearable ultrasound device. The display is also wearable, such as included as a portion of one or more of an eyepiece, headband, or glasses wearable by a user, the one or more of an eyepiece, headband, or glasses configured to present an image to the user without obstructing an entirety of a field of view of the user. In an example, the digital communication circuit comprises a wired, or optical, digital communication circuit, and a count of conductors included in a wired electrical interconnection between the digital communication circuit and the control circuit is less than a count of ultrasound transducer elements included in the transducer array. In an optically-coupled system, a multi-element cable including electrical and optical signal pathways can be used, such as using electrically conductive elements to supply power and using optical elements to provide digital data transfer.

In an example, such as alternatively or in addition to a wired digital communication circuit, the digital communication circuit comprises a first wireless communication circuit, and the display and control circuit are housed by a second housing, the second housing including second wireless communication circuit configured to communicatively couple the control circuit to the ultrasound transmit and receive circuit via the first wireless communication circuit.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally and example that can include an ultrasound imaging system, such as including a wearable ultrasound device.

FIG. 2A and FIG. 2B illustrate examples, generally, that can include an ultrasound imaging system, such as including a wearable ultrasound device having a wired or wireless communicative coupling to a display.

FIG. 3A illustrates generally an example that can include a wearable ultrasound device configured to insonify a tissue region below a housing, such as including insonification in a region extending radially outward from a surface of the housing.

FIG. 3B illustrates generally an example that can include a wearable ultrasound device such as located along or at the tip of a distal extension from a hand of user.

FIG. 4 illustrates generally a technique, such as a method, that can include identifying an anatomical target using a wearable ultrasound device.

FIG. 5 illustrates generally an illustrative example of an anatomical target, such as an intrathoracic artery, that can be located, such as using a wearable ultrasound device.

FIG. 6 illustrates generally a diagram of a system, such as upon which one or more examples (e.g., discussed methodologies) can be implemented (e.g., executed).

### DETAILED DESCRIPTION

The present inventors have recognized, among other things, a pressing need to find approaches that save time, money and improve patient outcomes in an environment that is moving towards increased scrutiny over total healthcare expenditures, such as involving capped procedure costs and incentives for reducing cost, along with demonstrating high quality (e.g. measured by low readmission rate). When feasible, a coronary artery bypass graft (CABG) procedure involves isolating a portion of the internal thoracic artery (ITA)—also known as the internal mammary artery (IMA)—and connecting this to the coronary artery distal of an occlusion. The ITA isolation process is generally conducted methodically using direct visual guidance and sequential fine passes of an electrocautery knife. Using a functioning artery in this manner has improved long term outcome statistics (e.g., providing a reduced rate of re-occlusion) as compared to use involving harvested vein vessels (such as the saphenous vein from the leg).

In multi-vessel disease, it is generally a practice to bypass one coronary vessel using the ITA and to bypass one or more additional vessels using vein grafts. Additionally, there is growing interest in “hybrid” procedures involving one CABG procedure combined with one or more PCI procedures.

In the absence of use of techniques and apparatus as described herein, an ITA harvest procedure commonly takes, as an illustrative example, 8 minutes to up to 20 minutes, or even a greater duration, depending on surgeon experience level and patient habitus. For example, the ITA may be obscured by pleura, fat and muscle. Additionally, since CABG bypass graft anastomosis is usually performed on an arrested heart, with the patient on cardiopulmonary bypass, there is a quality of life contribution to be gained by reducing the “on pump” duration. In this context, the proposed ultrasound apparatus and techniques described herein can be used to more definitively identify the optimal point of anastomosis along a vessel's length or finding the bypass target vessel if obscured by overlying fat.

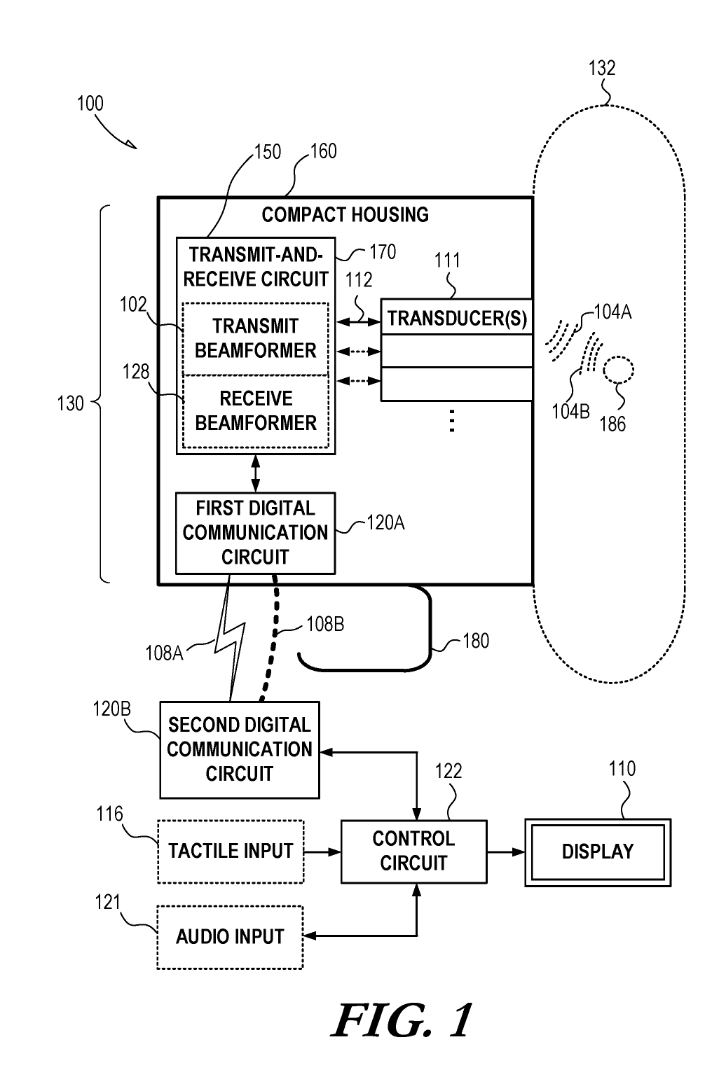

FIG. 1 illustrates generally and example that can include an ultrasound imaging system 100, such as including a wearable ultrasound device 130. The wearable ultrasound device 130 can include a compact housing 160, such as sized and shaped to be lifted and manipulated by as few as a single finger of a user. The compact housing can house “front end” circuitry of the ultrasound imaging system 100, such as transmit-and-receive circuit 170. The transmit-and-receive circuit can include one or more beamformers such as a transmit beamformer 102 or a receive beamformer 128. Other circuitry such as analog-to-digital converters and power amplifier circuits can also be located in the compact housing, such as co-integrated as a portion of a commonly-shared integrated circuit or integrated circuit package. The transmit-and-receive circuit 170 can be coupled to an ultrasound transducer array 111, such as including two or more transducer elements. As an illustrative example, such an array can include eight ultrasound transducer elements, such as arranged in a linear array, or another count, such as 64 transducer elements, such as arranged in a two-dimensional array.

The transducer elements can be controlled to emit and couple an acoustic transmission signal 104A in an ultrasonic frequency range. Ultrasound echoes 104B can be generated by scattering or reflections from an object such as an anatomical target 186. The echoes 104B can be received by the same transducer array 111 as was used for generation of the transmission signal 104A, or a different group of elements from the array 111 can be used, or even another separate receive array can be used.

Received ultrasound echoes can be converted to electrical signals in analog form, and such signals can be referred to as “RF” or “radio frequency” signals, such as coupled via an analog interface 112 to the transmit-and-receive circuit 170. A channel count of analog channels in the interface 112 generally corresponds to a count of ultrasound transducer elements in the array 111. Accordingly, the present inventors have recognized that reducing a channel count, such as by digitizing the RF information, enables analog “front end” processing and digitization to be achieved on-board the wearable ultrasound device 130, allows a lower-complexity (e.g., fewer physical electrical interconnects or a lower-bandwidth communication channel) to be used to send digital information indicative of received ultrasound echoes to a display located elsewhere. In this manner, the wearable ultrasound device 130, including front-end processing circuitry, can be single-use or disposable. For example, the wearable ultrasound device 130 can include a mechanical anchor such as a clip 180, such as can be configured to securely anchor the wearable ultrasound device 130 to a user's hand, such as to a fingertip.

The wearable ultrasound device 130 can also include a communication circuit, such as a first digital communication circuit 120A. The communication circuit can include one or more of wired or wireless communication capability, such as conforming to an accepted standard. Such standards can include one or more of wired or wireless networking standards, or a wired serial communication standard, as illustrative examples. A wireless communicative coupling 108A or a wired communicative coupling 108B can be provided, such as to other portions of the ultrasound system 100 located elsewhere. For example, a digital representation indicative of received ultrasound echo information can be received from the first digital communication circuit 120A by a second digital communication circuit 120B. The system 100 can be partitioned, such as having a display 110 (e.g., a light-emitting diode display or liquid crystal display) located separately from the wearable ultrasound device 130.

The display 110 can be coupled to a control circuit 122, such as having a microprocessor-based or microcontroller-based architecture. In examples where the display 110 is located separately from the wearable ultrasound device 130, the wearable device can also include a control circuit. The control circuit 122 can receive inputs such as operational adjustments or commands from a user, such as using one or more of a tactile input 116, such as a touch-screen incorporated as a portion of the display 110 or one or more other inputs, or an audio input 121, such as to receive oral commands. Other types of tactile input 116 can be used, such as soft-keys, push-buttons, switches, membrane keypads, or rotary controls including digital (e.g., rotary encoder or rotary switch) or analog (e.g., potentiometer) controls.

The system 100 can be controlled by the control circuit 122, such as to dynamically adjust a focal distance or scan depth for ultrasound-based imaging. For example, the control circuit can provide or control scan conversion, wherein a digital representation of received ultrasound echo information is converted to information for display, such as providing a specified imaging mode (e.g., B-mode imaging, C-mode imaging, color Doppler, or color imaging that presents a Doppler-like presentation, such as blood motion imaging using speckle motion or image data decorrelation), and having a specified scan line count, pitch, or image acquisition rate. One or more of the transmit or receive beamformers 102 or 128 can be controlled to establish transmit pulses and perform acquisition of received echo information according to the selected imaging mode and other selected characteristics.

For example, imaging depth can be determined assuming a constant speed of sound in tissue (e.g., 1540 m/s or as desired or required) and then time of flight can be determined such that the returning echoes have a known origination locus within the targeted region 132 being imaged. Summed RF lines can be provided, such as by the receive beamformer 128, and further processing can be performed, such as after digitization and transmission of such summed RF line information via the first digital communication circuit 120A. For example, a principal components processor can be used to identify an anatomical feature of interest within a targeted imaging region 132. The principal components processor may be separate from and controlled by, or incorporated in the control circuit 122. The control circuit 122 can provide or can control a scan converter such that assembled information can then be provided to the display 110.

As illustrative examples, various hardware configurations can be used to implement the ultrasound system 100 shown in FIG. 1. In a first illustrative example, dimensions for a clip-able single-use transducer array, such as can be included in a wearable ultrasound device 130 can include an ultrasound aperture defined by an area of about 10 millimeter (mm) by 10 mm, such as housed or coupled to a housing having dimensions of about 14 mm by 14 mm. A thickness of such a housing can include a sufficient thickness to provide, for example, an acoustic backing and one or more integrated circuits. For example, two or more integrated circuits can be packaged in a stacked configuration. As an illustrative example, acoustic backing of about 1 to about 2 mm have been shown to be feasible in transesophageal applications. Accordingly, without being bound by theory, the present inventors have recognized, among other things, that an overall wearable device thickness (not including the mechanical anchor or clip) can be about 5 mm to about 7 mm, as in an illustrative example.

Acoustic operating frequency ranges for the ultrasound system 100 can include a frequency selected from a range of about 5 to about 10 megahertz (MHz), or even beyond 10 MHz, such as specified at least in part consistent with a desired imaging depth—such as a depth of about 30 mm). Wavelength element spacing can be specified to for consistency with non-steered imaging operation, such as 32 elements spaced at 0.3 mm (e.g., for 5 MHz operation)) or 64 elements spaced at 0.15 mm (e.g., for 10 MHz operation). Such examples are illustrative, and other counts of transducers can be used, having a different specified element spacing. The transducer elements can include a high dielectric constant piezoceramic, such as CTS 3203HD available from CTS Communications Components (Bloomingdale, Ill., USA), or PZT-5H. One or more other materials can be used, such as single crystal piezoelectric materials, or “composite” transducer materials comprising blends of piezoelectric ceramic (or single crystal) in an ordered polymer matrix (e.g. “1-3” Piezoelectric-epoxy composite materials). The transducer elements can also be non-piezoelectric—such a capacitive Micromachine Ultrasound Transducer (cMUT) built directly upon a silicon substrate.

The transducer materials can be formed into discrete independently operable transducer elements. The elements can have one, two, or more nominally quarter-wavelength matching layers located on their acoustically-emitting (e.g., “front”) surface. The transducer beam can be constrained in an elevational (e.g., beam slice) dimension, such as using a filled “room temperature vulcanization” (RTV) silicone. Generally, RTV silicone can be filled with alumina or silicon particles to achieve a higher density and lower acoustic propagation velocity so as to provide one or more of an acoustic match or lens focusing qualities, with an appropriate elevational lens. The lens curvature can be established using the Lensmaker's Equation knowing the desired focal depth, and propagation velocities in each of the lens material and an imaging target medium such as tissue. As illustrative examples, propagation velocities of sound can be about 1000 meters per second (m/s) in filled RTV and about 1540 m/s in tissue.

A lossy backing material can be provided to sink acoustic energy propagating in a rearward direction. The backing can include a “soft” polymer—such as a relatively-low durometer epoxy that can be filled with a blend of particles. Such particles can include one or more of alumina, silica (such as including glass spheres or microballoons), metal, or metal oxide particles. As an illustratively example, particles such as tungsten metal—typically in 1-3 micron particle size—can be used. U.S. Pat. 5,297,553 mentions an acoustically lossy backing, and is hereby incorporated herein by reference.

Various imaging modes can be supported by the ultrasound system 100. Such modes can include B-mode, “blood flow.” For example, blood can be imaged using image speckle motion or image data decorrelation giving the appearance of a color Doppler presentation. User-controllable inputs to the ultrasound system 100 can include one or more of brightness or gain control, imaging depth setting, and imaging mode control (B-mode, blood flow, CW Doppler such as having an audible feedback signal for the user).

As an illustrative example, a 1D imaging array and system can include use of a 5 MHz ultrasound center frequency, and the transducer element array 111 can include a 32 element array having 0.3 mm pitch, or another configuration can be used, such as tailored by an specialized vendor such as Vermon S.A. (Tours, France). For improved resolution, and bearing in mind that only minimal imaging depth may be needed for a particular application, a 10 MHz or higher frequency can be used. In this illustrative example, there could be approximately 64 elements spaced on a 0.15 mm pitch. Yet higher frequencies, such as 12-15 MHz, are also usable—but with the added complexity and associated expensive involved in finer spatial sampling and more elements/channels to provide around a 10 mm aperture. Materials for one or more of the housing or clip 180 can include materials having a size and stiffness to allow the clip 180 to securely anchor the wearable ultrasound device 130 to a finger of a clinician (e.g., a surgeon). For example, a polymer material such as Radel R5500 polysulphone available from Westlake Plastics (Lenni, Pa., USA) can be used. Integrated ultrasound “front-end” electronics are available from Texas Instruments (TI) and other integrated circuit vendors (e.g. Maxim). As an illustrative example, TI provides components having support for eight channels. Accordingly, a 32 channel implementation may use four of each of following, available from Texas Instruments Inc., (Dallas, Tex., USA):

TI AFE 5805 8 channel analog front end

TI LM96570 8 channel transmit beamformer

TI 8 channel transmit/receive switch

Other circuitry included as a portion of the wearable ultrasound device 130 can include a processor or control circuitry, such as a field-programmable gate array (FPGA) and a digital signal processor (e.g., a TI C64-series digital signal processor that can provide a receive beamformer 128).

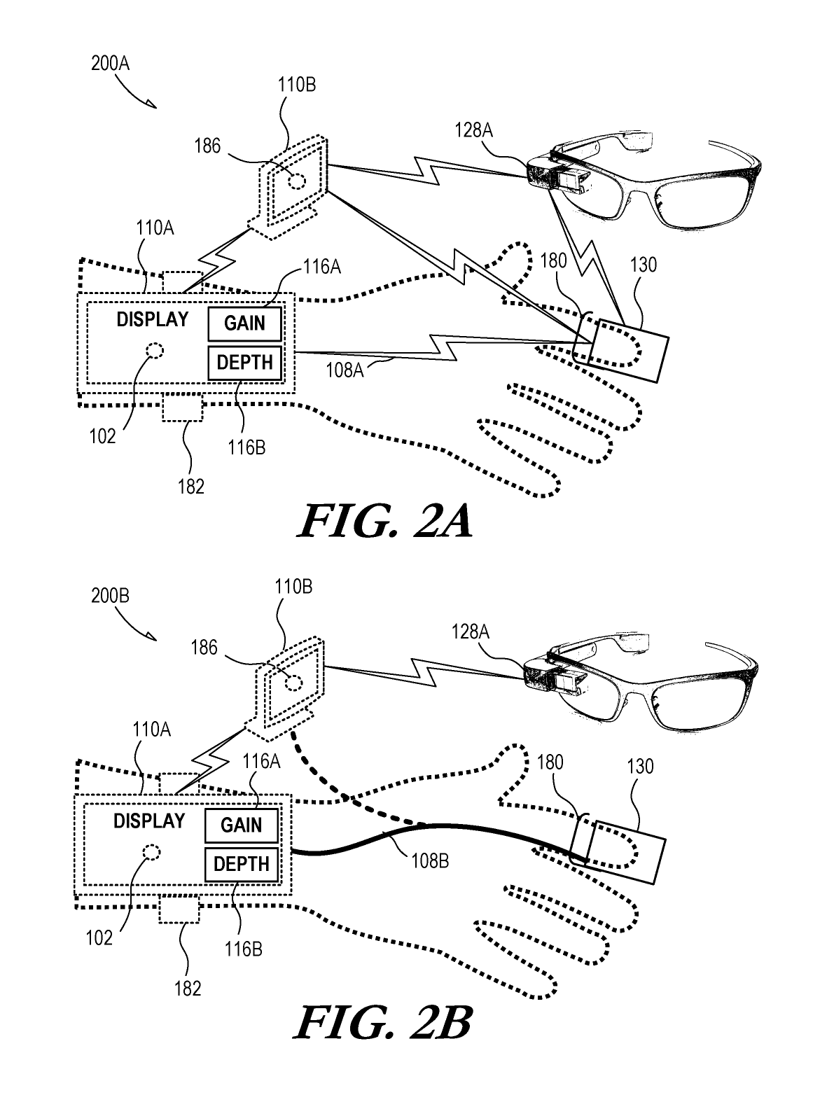

As mentioned above, the system 100 can be partitioned, such as shown illustratively in FIG. 2A and FIG. 2B. Referring back to FIG. 1, the display 110 can be located separately from the wearable ultrasound device 130 and compact housing 160. For example, the display can also be a portion of a wearable assembly, or a fixed “base station” located elsewhere in the operating environment of the system 100. The display device can include or can be coupled to an “Open Multimedia Application Platform” (OMAP) microprocessor, such as available from Texas Instruments Inc. (Dallas, Tex., USA), such as including a TI OMAP35-series for use as a “back end” processor separated from a single-use or disposable wearable device 130. For example, the control circuit 122 can include an OMAP processor or other microprocessor or microcontroller.

The control circuit 122 can obtain wirelessly-coupled or wire-coupled information indicative of received ultrasound echo information, such as a serial representation of digitized beamformed data, and the can convert such information into a parallel digital data structure. Such parallel data can be envelope detected (e.g., to obtain a signal magnitude) and scan converted for display. The acquisition format can be rectilinear (not requiring steering), and accordingly, the scan conversion to final display resolution can be a two-dimensional (2D) interpolation.

A “flow mode,” such as for use in identifying a vessel or lumen boundary in a targeted tissue region 132, can function by detecting rapidly-changing signals between sequential acquisitions. Loss of mathematical correlation (or even simply signal differences) can be regarded as an indication of blood flow. Signals relating to loss of correlation, or signal difference, such as difference exceeding a specified threshold, can be mapped as color pixels that thereby highlight regions of blood flow. The nature of the detected flow, while not quantitative, is sufficient to provide an estimate of a size of the vessel, and can assist in providing a visual indication to a user of whether flow is pulsatile (e.g., arterial) or approximately continuous (e.g., venous). For example, such indications can be useful to a surgeon in accessing the presence and significance of detected blood vessels during a procedure such as CABG.

Imaging performance can be improved, if necessary, using one or more techniques. For example, images can be acquired at different ultrasound center frequencies and compounded (summed), such as compounded post-envelope-detection to obtain a reduced speckle image. In another approach, nonlinear gray-scale-mapping can be used to accentuate different image features or to de-emphasize artifacts. In this context “nonlinear” means other than straight line, such as using a mapping having an “S” shape. Blood flow visualization can be improved, such as by administering microbubble contrast agent intravenously into the patient. While not required, use of an echogenic contrast agent can turn a dark-appearing low-echo signal, such as of a vessel, into a bright high-echo region.

In another illustrative example of a hardware configuration for the ultrasound system 100, one or more devices, systems, compositions, non-transitory computer readable media, or techniques such as methods of various examples described herein may use aspects disclosed in one or more of the following references, applications, publications, and patents, each of which is hereby incorporated by reference herein in its entirety (and which are not admitted to be prior art with respect to the present invention by inclusion in this section):

a. U.S. Pat. No. 8,057,392, Hossack, et al., “Efficient Architecture for 3D and Planar Ultrasonic Imaging-Synthetic Axial Acquisition and Method Thereof”, Nov. 15, 2011.

b. U.S. Pat. No. 7,750,537, Hossack, et al., “Hybrid Dual Layer Diagnostic Ultrasound Transducer Array”, Jul. 6, 2010.

c. U.S. Pat. No. 7,699,776, Walker, et al., “Intuitive Ultrasonic Imaging System and Related Method Thereof”, Apr. 20, 2010.

d. U.S. Pat. No. 7,402,136, Hossack, et al., “Efficient Ultrasound System for Two-Dimensional C-Scan Imaging and Related Method Thereof”, Jul. 22, 2008.

e. U.S. Patent Application Publication No. 2012/0053460 A1, Blalock, et al., “Ultrasound Imaging Beam-Former Apparatus and Method” Mar. 1, 2012.

f. U.S. Patent Application Publication No. 2012/0029356 A1, Hossack, et al., “Efficient Architecture for 3D and Planar Ultrasonic Imaging-Synthetic Axial Acquisition and Method Thereof”, Feb. 2, 2012.

g. U.S. Patent Application Publication No. 2011/0137175 A1, Hossack, et al., “Tracked Ultrasound Vessel Imaging”, Jun. 9, 2011.

h. U.S. Patent Application Publication No. 2010/0312106 A9, (Corrected Publication), Blalock, et al., “Ultrasound Imaging Beam-Former Apparatus and Method”, Dec. 9, 2010.

i. U.S. Patent Application Publication No. 2010/0268086 A1, Walker, et al., “Intuitive Ultrasonic Imaging System and Related Method Thereof”, Oct. 21, 2010.

j. U.S. Patent Application Publication No. 2010/0063399 A1, Walker, et al., “Front End Circuitry for Imaging Systems and Methods of Use”, Mar. 11, 2010.

k. U.S. Patent Application Publication No. 2009/0048519 A1, Hossack, et al., “Hybrid Dual Layer Diagnostic Ultrasound Transducer Array”, Feb. 19, 2009.

l. U.S. Patent Application Publication No. 2007/0016044 A1, Blalock, et al., “Ultrasonic Transducer Drive”, Jan. 18, 2007.

m. U.S. Patent Application Publication No. 2007/0016022 A1, Blalock, et al., “Ultrasound Imaging Beam-Former Apparatus and Method”, Jan. 18, 2007.

n. U.S. Patent Application Publication No. 2006/0100516 A1, Hossack, et al., “Efficient Architecture for 3D and Planar Ultrasonic Imaging-Synthetic Axial Acquisition and Method Thereof”, May 11, 2006.

o. U.S. Patent Application Publication No. 2006/0052697 A1, “Efficient Ultrasound System for Two-Dimensional C-Scan Imaging and Related Method Thereof”, Mar. 9, 2006.

p. U.S. Patent Application Publication No. 2005/0154303 A1, “Intuitive Ultrasonic Imaging System and Related Method Thereof”, Jul. 14, 2005.

In this manner, a tightly integrated pulser, transmit/receive isolation, analog front end, digitization and beamforming implementation can be performed such as on-board a wearable ultrasound device 130 as shown in FIG. 1. An output of beamformed received signals are thereby at an appropriately low signal bandwidth to facilitate being communicated via a digital communication interface, such as a low-conductor-count wired interface. In an illustrative example, the receive circuitry of the wearable ultrasound device can be operated in a manner to obtain complex-valued samples without requiring a coherent demodulation scheme, such as using a direct-sampled in-phase/quadrature (DSIQ) sampling technique.

FIG. 2A and FIG. 2B illustrate examples, generally, that can include an ultrasound imaging system 200A or 200B, such as including a wearable ultrasound device 130 having a wired or wireless communicative coupling to a display. In FIG. 2A, the wearable ultrasound device 130 can include one or more wireless communication circuits, such as to provide a wireless digital communicative coupling 108A with one or more other devices such as a wearable display 110A, or a “base station” display 110B. Such digital communication can be performed according to a short-range communication standard or local-area wireless networking protocol.

Referring to FIG. 2B, the wearable ultrasound device 130 can provide a wired communicative coupling 108B to one or more of the wearable display 110A or another display 110B. The examples of FIG. 2A and 2B are not mutually exclusive. For example, the wearable ultrasound device 130 can include both wired and wireless digital communication circuits. For example, according to one of the illustrative hardware implementations discussed above, an interface between a digital signal processor (DSP), such as included as a portion of the wearable ultrasound device 130, and an OMAP device, such as included as a portion of a display or base station, can include uses a serial digital interface. Such a serial digital interface can include a Serial Communications Interface (SCI) supported by the TI C64-series of devices. In this manner, an interface cable providing a communicative coupling 108 can be thin and light, and need not carry conductors corresponding to each of the elements in the transducer array. If information indicative of received echo information from the transducer array is beamformed (preferred), highly compressed, or otherwise multiplexed, a serial data interface can be used. Although probably not necessary, it is also possible to have a hybrid of parallel and serial communication. If a single serial channel is insufficient, then multiple serial channels, such as respectively carrying the multiplexed, or summed, components of several transducer channels, can be operated in parallel so that all data from across the entire array is accommodated.

Referring generally to the examples of FIG. 2A and FIG. 2B, the display 110A can include an anchor 182, such as a band, to secure the display 110A to a user's hand. In addition, or alternatively, the user can be presented with imaging information using another display located elsewhere. For example, the display may take the form of a generic video display 110B as mounted in modern operating room (OR) settings (e.g. the displays associated with laparoscope or other minimally invasive surgical instruments) or otherwise provided by a desktop computer, a laptop computer, or a tablet device. As another illustrative example, the display data can be formed in to a “video call” and communicated to a smart device such as an Android or iOS-based communication device (a smart phone or, again, a tablet device). In yet another example, display data such as in the form of a “video call” or otherwise transmitted wireless can be coupled to a display included as a portion of one or more of an eyepiece, headband, or glasses wearable by the user. For example, a Google Glass™ (Google, Inc., Mountain View, Calif., USA) style of device can include a display that can present an image representation of ultrasound echo information obtained using the wearable ultrasound device 130, but such an example is illustrative and other configurations can be used.

One or more of the first display 110A or the second display 110B can include a second housing independent of the wearable ultrasound device 130. The second housing can house elements of the system 100 as shown in FIG. 1 such as the control circuit, inputs, and one or more communication circuits.

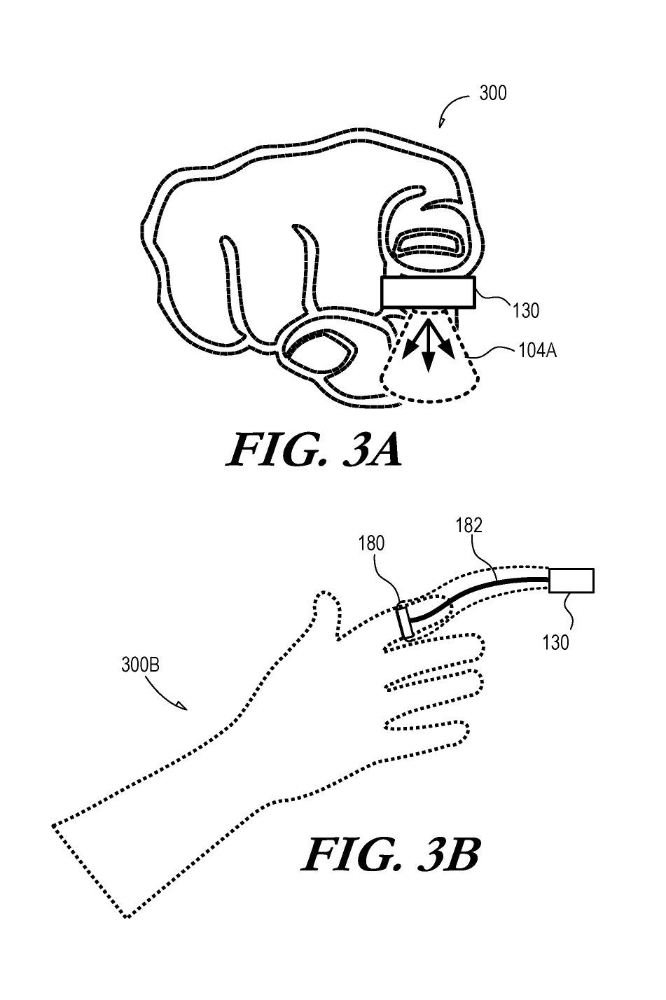

FIG. 3A illustrates generally an example that can include a wearable ultrasound device 130 configured to insonify a tissue region below a housing, such as including insonification in a region 104A extending radially outward from a surface of the housing. In this manner, a user such as clinician performing a medical procedure (e.g., surgery) can scan a region below a fingertip, or a region below an imaging transducer array coupled to a fingertip as shown illustratively in FIG. 3B, which illustrates generally an example that can include a wearable ultrasound device 130 such as located along or at the tip of a distal extension 182 from a hand of user. The distal extension 182 or “extender” can extend in a direction beyond a length of a user's finger, such as extending to a distance of about 150 mm. The distal extension 182 can have a cross sectional area smaller than a corresponding area of the housing of the ultrasound device 130, or the extender can have a cross section similar to a finger to permit use of the same anchoring clip as can be used if the ultrasound device 130 is located directly on a finger of a user without use of an extension. In an example, the distal extension 182 can include a clip 180 or other anchor (such as circumferential section) to securely anchor the extension 182 to a fingertip of the user. In this manner, regions can be probed that extend beyond the fingertip of the user, and such extension may broaden a range of suitable applications for an ultrasound system including the wearable ultrasound device 130. The distal extension 182 can be one or more of articulated (such as including one or more joints) or made malleable. In this manner, the extension 182 can be manipulated, such as bent to suit a particular surgical or imaging application or anatomy. As an illustrative example, a soft polymer or rubber (e.g. silicone rubber) with a malleable wire core can be used, such as an electrical-grade solid copper wire (e.g., a 12 gauge copper wire).

FIG. 2A, FIG. 2B, FIG. 3A, and FIG. 3B are illustrative examples. Other configurations can be used, such as including features mentioned in the following examples:
Additional or various controls can be included as a portion of the wearable ultrasound device assembly itself. The controls may include sealed membrane switches or other inputs, such as to control “brightness up/down” and “depth up/down” or to provide others controls and functions. For example, the user may want to control the features or parameters without a) going outside sterile field or b) asking an assistant to operate the machine on behalf of the user.
Various clip configurations can be used, such as can include a plastic clip, a strap, or other attachment techniques. For example, one approach may include a tube-over-finger configuration (as mentioned in relation to the distal extension 182 for example, or other configurations such as a split tube configuration or a “between fingers” configuration.
One or more of the housing of the wearable ultrasound device, or even an entirety of the wearable ultrasound device can be disposable, such as to provide a “single use” assembly for a particular procedure or other instance of use without requiring re-use or sterilization.

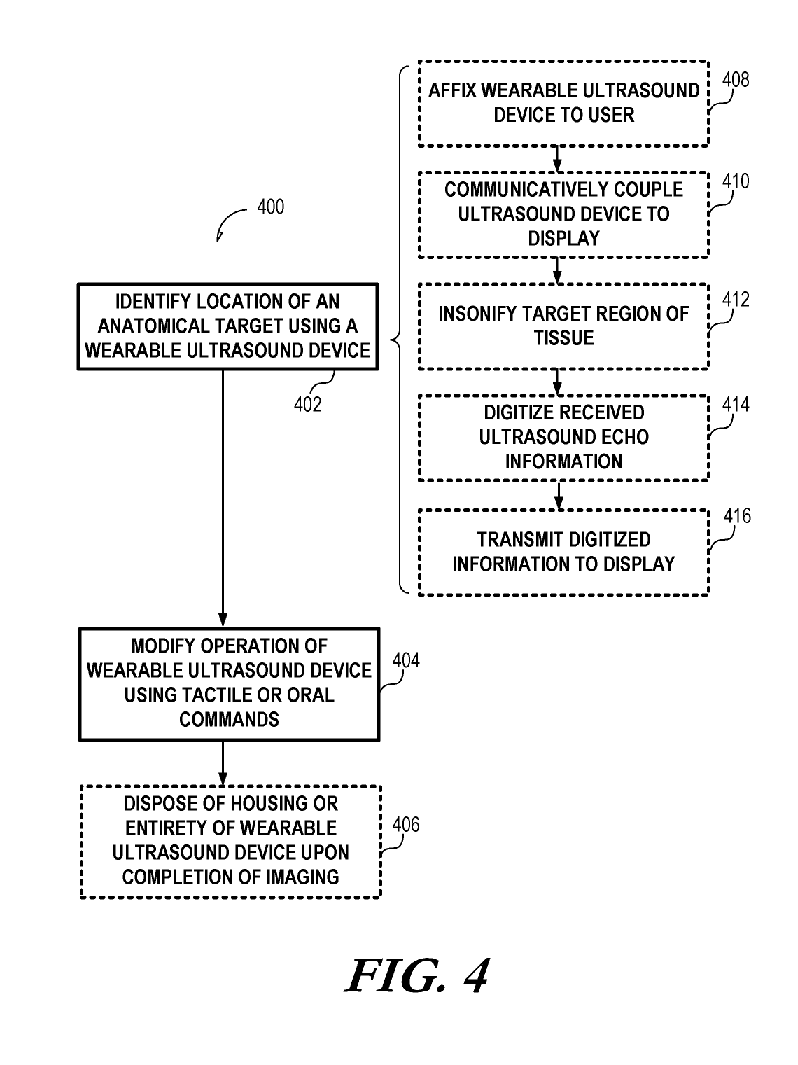

FIG. 4 illustrates generally a technique 400, such as a method, that can include at 402 identifying an anatomical target using a wearable ultrasound device. For example, at 408, the wearable ultrasound device can be affixed to a user, such as clipped to a fingertip of a user. At 410, the wearable ultrasound device can be communicatively coupled to a display, such as using one or more of a wired or wireless communication link. At 412, a target region of tissue can be insonified, such as using transmitted pulses provided by the wearable ultrasound device. At 414, received echo information, such as received by the wearable ultrasound device, can be digitized. Other operations can be performed on received signals, such as one or more of beamforming, averaging, or other processing. At 416, digitized information can be transmitted to a display, such as a display included as a portion of another wearable device or a base station. At 404, operation of the wearable ultrasound device can be modified, such as using one or more of tactile (keys, knobs, buttons, or a touchscreen, as illustrative examples) or oral commands received by an ultrasound system. Optionally, at 406, a housing or an entirety of the wearable ultrasound device can be discarded, such as upon completion of an imaging instance. For example, an imaging instance can correspond to an imaging session during a surgical procedure.

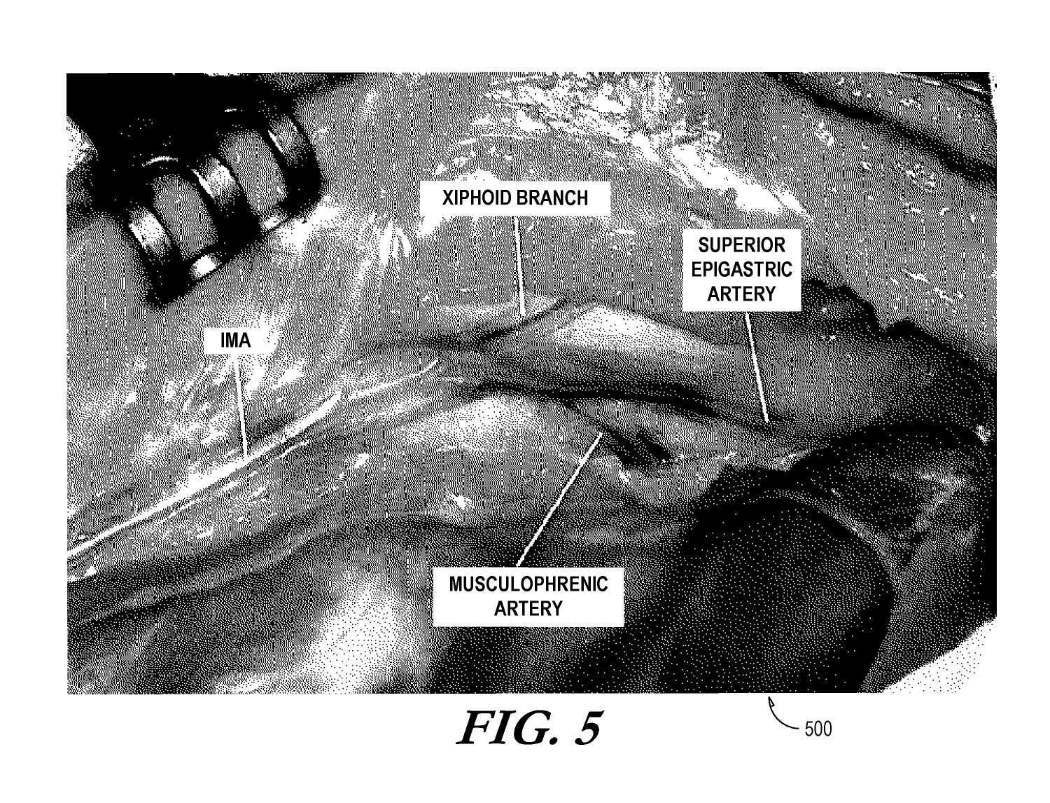

FIG. 5 illustrates generally an illustrative example 500 of an anatomical target, such as an intrathoracic artery or internal mammary artery (IMA), that can be located, such as using a wearable ultrasound device. In this context, the proposed ultrasound apparatus and techniques described herein can be used to more definitively identify the optimal point of anastomosis along a vessel length or to assist in finding a bypass target vessel if obscured by overlying fat.

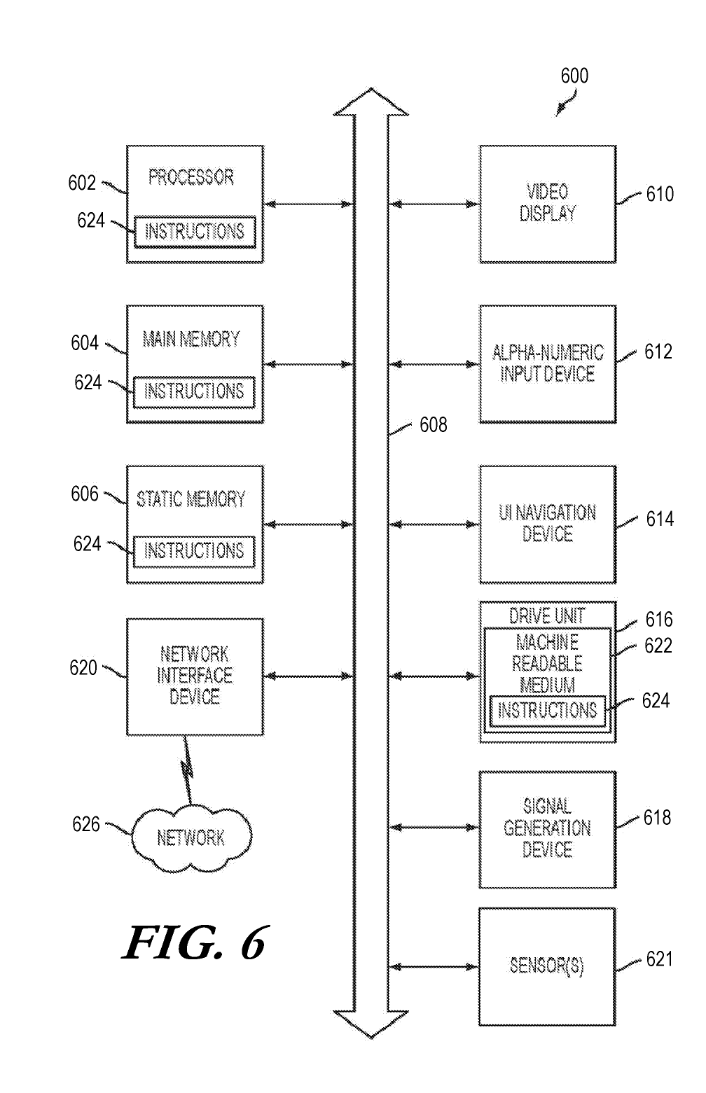

FIG. 6 illustrates generally a diagram of a system 600, such as upon which one or more examples (e.g., discussed methodologies) can be implemented (e.g., executed). Examples of machine 600 can include logic, one or more components, or circuits. Circuits are tangible entities configured to perform certain operations. In an example, circuits can be arranged (e.g., internally or with respect to external entities such as other circuits) in a specified manner. In an example, one or more computer systems (e.g., a standalone, client, or server computer system) or one or more hardware processors (processors) can be configured by software (e.g., instructions, an application portion, or an application) as a circuit that operates to perform certain operations as described herein. In an example, the software can reside (e.g., be stored or otherwise encoded) using a non-transitory machine-readable medium. In an example, the software, when executed by the underlying hardware of the circuit, causes the circuit to perform the certain operations.

A circuit can include dedicated circuitry or logic that is specifically configured to perform one or more techniques such as discussed above, such as including a special-purpose processor, a field programmable gate array (FPGA) or an application-specific integrated circuit (ASIC). In an example, a circuit can include programmable logic (e.g., circuitry, as encompassed within a general-purpose processor or other programmable processor) that can be temporarily configured (e.g., by software) to perform the certain operations. It is possible to implement a circuit in a permanent, application-specific manner, or in temporarily configured circuitry (e.g., configured by software).

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Examples described herein (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 600) and software architectures that can be deployed in example embodiments.

In an example, the machine 600 can operate as a standalone device or the machine 600 can be connected (e.g., networked) to other machines. In a networked deployment, the machine 600 can operate in the capacity of either a server or a client machine in server-client network environments. In an example, machine 600 can act as a peer machine in peer-to-peer (or other distributed) network environments. The machine 600 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 600. Further, while only a single machine 600 is illustrated, the term “machine” shall also be taken to include any collection of machines that individually or jointly execute a set (or multiple sets) of instructions to perform any one or more of the methodologies discussed herein.

In an example, the machine (e.g., computer system) 600 can include a processor 602 (e.g., a central processing unit (CPU), a graphics processing unit (GPU) or both), a main memory 604 and a static memory 606, some or all of which can communicate with each other via a bus 608. The machine 600 can further include a display unit 610, an alphanumeric input device 612 (e.g., a keyboard), and a user interface (UI) navigation device 611 (e.g., a mouse). In an example, the display unit 810, input device 617 and UI navigation device 614 can be a touch screen display. The machine 600 can additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The wearable ultrasound device mentioned in relation to FIG. 1, FIG. 2A, FIG. 2B, FIG. 3A, or FIG. 3B need not include the elements described in FIG. 6, or arranged as in FIG. 6, but the systems mentioned elsewhere can include portions of FIG. 6. For example, a display unit in an operating theater may include the mechine 600 of FIG. 6, as an illustrative example.

The storage device 616 can include a machine readable medium 622 on which is stored one or more sets of data structures or instructions 624 (e.g., software) embodying or utilized by any one or more of the methodologies or functions described herein. The instructions 624 can also reside, completely or at least partially, within the main memory 604, within static memory 606, or within the processor 602 during execution thereof by the machine 600. In an example, one or any combination of the processor 602, the main memory 604, the static memory 606, or the storage device 616 can constitute machine readable media.

While the machine readable medium 622 is illustrated as a single medium, the term “machine readable medium” can include a single medium or multiple media (e.g., a centralized or distributed database, and/or associated caches and servers) that configured to store the one or more instructions 624. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 624 can further be transmitted or received over a communications network 626 using a transmission medium via the network interface device 620 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Examples of ultrasound systems, such as including examples shown in portions or entireties of one or more of FIG. 1, FIG. 2A, FIG. 2B, FIG. 3A, or FIG. 3B can include a system 600 or a portion of the system 600 as shown in FIG. 6. For example, one or more of a display assembly or a wearable ultrasound device can include one or more processor circuits, such as coupled to a memory or other storage, the processor circuit configured to perform a method or technique using instructions stored in the memory or other storage.

### Various Notes & Examples

Each of these non-limiting examples described in this document can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An ultrasound imaging system, comprising:
a wearable ultrasound device including:
an ultrasound transducer array;
a wearable clip mechanically coupled to the transducer array;
a housing including:
an ultrasound transmit-and-receive circuit electrically coupled to the transducer array; and
a digital communication circuit coupled the ultrasound transmit-and-receive circuit, the digital communication circuit configured to provide a digital representation indicative of receive ultrasound echo information;
wherein the wearable clip is configured to securely anchor the wearable ultrasound device assembly to a finger of a user; and
wherein the ultrasound transducer array, wearable clip, and housing are sized and shaped to be lifted and manipulated by as few as a single finger of a user.

2. The ultrasound imaging system of claim 1, comprising a display coupled to a control circuit to receive the digital representation indicative of received ultrasound echo information.

3. The ultrasound imaging system of claim 2, wherein the display is separate from the housing of the wearable ultrasound device.

4. The ultrasound imaging system of claim 3, wherein the display is also wearable.

5. The ultrasound imaging system of claim 4, wherein the display is included as a portion of one or more of an eyepiece, headband, or glasses wearable by a user, the one or more of an eyepiece, headband, or glasses configured to present an image to the user without obstructing an entirety of a field of view of the user.

6. The ultrasound imaging system of claim 2, wherein the digital communication circuit comprises a wired digital communication circuit, and wherein a count of conductors included in a wired electrical interconnection between the digital communication circuit and the control circuit is less than a count of ultrasound transducer elements included in the transducer array.

7. The ultrasound imaging system of claim 2, wherein the digital communication circuit comprises a first wireless communication circuit, and
wherein the display and control circuit are housed by a second housing, the second housing including second wireless communication circuit configured to communicatively couple the control circuit to the ultrasound transmit and receive circuit via the first wireless communication circuit.

8. The ultrasound imaging system of claim 2, wherein the control circuit is coupled to an audio input circuit; and
wherein the control circuit is configured to establish or alter a mode of operation of the displayed ultrasound information in response to an oral command from a user.

9. The ultrasound imaging system of claim 1, wherein the ultrasound transducer array is arranged to insonify a region extending radially outward from a finger tip when the wearable clip is anchored to a finger of a user.

10. The ultrasound imaging system of claim 1, wherein the ultrasound transducer array of claim 1, wherein the ultrasound transducer array is coupled to the wearable clip using an elongate member to extend a reach of a finger of a user when the wearable clip is anchored to the finger of the user.

11. The ultrasound imaging system of claim 10, wherein the elongate member is one or more of articulated, malleable, or flexible, to allow repositioning of the ultrasound transducer array relative to a finder of the user to which the

12. The ultrasound imaging system of claim 1, wherein the wearable ultrasound device assembly comprises a housing having a sterile exterior surface.

13. The ultrasound imaging system of claim 12, wherein one or more of the housing or an entirety of the wearable ultrasound device assembly is disposable.

14. The ultrasound imaging system of claim 1, wherein a count of ultrasound transducer elements included in the array is selected from a range of 8 to 64 elements.

15. The ultrasound imaging system of claim 1, wherein the ultrasound transmit-and-receive circuit is configured to acquire complex-valued samples of reflected ultrasound echoes using a direct-sampled in-phase and quadrature (DSIQ) technique.

16. An ultrasound imaging system, comprising:
a wearable ultrasound device including:
an ultrasound transducer array;
a wearable clip mechanically coupled to the transducer array;
a housing including:
an ultrasound transmit-and-receive circuit electrically coupled to the transducer array; and
a digital communication circuit coupled the ultrasound transmit-and-receive circuit, the digital communication circuit configured to provide a digital representation indicative of receive ultrasound echo information; and
a display coupled to a control circuit to receive the digital representation indicative of received ultrasound echo information;
wherein the display is separate from the housing of the wearable ultrasound device,
wherein the digital communication circuit comprises a wired digital communication circuit, and wherein a count of conductors included in a wired electrical interconnection between the digital communication circuit and the control circuit is less than a count of ultrasound transducer elements included in the transducer array,
wherein the wearable clip is configured to securely anchor the wearable ultrasound device assembly to a finger of a user,
wherein the ultrasound transducer array, wearable clip, and housing are sized and shaped to be lifted and manipulated by as few as a single finger of a user,
wherein one or more of the housing or an entirety of the wearable ultrasound device assembly is disposable.

17. A method, comprising:
identifying a location of an anatomical target using ultrasound imaging, the ultrasound imaging provided at least in part using a wearable ultrasound device, the wearable ultrasound device including:
an ultrasound transducer array;
a wearable clip mechanically coupled to the transducer array;
a housing including:
an ultrasound transmit-and-receive circuit electrically coupled to the transducer array; and
a digital communication circuit coupled the ultrasound transmit-and-receive circuit, the digital communication circuit configured to provide a digital representation indicative of receive ultrasound echo information;
wherein the wearable clip is configured to securely anchor the wearable ultrasound device assembly to a finger of a user; and
wherein the ultrasound transducer array, wearable clip, and housing are sized and shaped to be lifted and manipulated by as few as a single finger of a user;
modifying operation of the wearable ultrasound device using one or more of a tactile user input located on or near the housing when the housing is within a sterile field, or in response to oral command from the user when the housing is within a sterile field.

18. The method of claim 17, comprising disposing of the housing or an entirety of the wearable ultrasound device upon completion of an imaging instance.

19. The method of claim 17, wherein the ultrasound transducer array is arranged to insonify a region extending radially outward from a finger tip when the wearable clip is anchored to a finger of the user.

20. The method of claim 17, wherein the ultrasound transducer array is coupled to the wearable clip using an elongate member to extend a reach of a finger of a user when the wearable clip is anchored to the finger of the user.

21. The method of claim 17, comprising presenting imaging information representative of receive ultrasound echo information using a display coupled to a control circuit, the control circuit configured to receive the digital representation indicative of received ultrasound echo information.

22. The method of claim 21, wherein presenting the imaging information includes presenting information indicative of an anatomical target including a blood vessel.

23. The method of claim 22, wherein the blood vessel comprises an internal thoracic artery imaged using the wearable ultrasound device.

24. The method of claim 21, wherein the display is separate from the housing of the wearable ultrasound device.

25. The method of claim 22, wherein the display is also wearable.

26. The method of claim 25, wherein the display is included as a portion of one or more of an eyepiece, headband, or glasses wearable by a user, the one or more of an eyepiece, headband, or glasses configured to present an image to the user without obstructing an entirety of a field of view of the user.

27. The method of claim 17, wherein the ultrasound transmit-and-receive circuit is configured to acquire complex-valued samples of reflected ultrasound echoes using a direct-sampled in-phase and quadrature (DSIQ) technique.
